(19) United States
(12) Patent Application Publication
(10) Pub. No.: US 2005/0031710 A1
(43) Pub. Date: Feb. 10, 2005

(54) Method of personal care and cosmetic product preparation and composition using human blood type

(76) Inventor: Peter James D'Adamo, New Canaan, CT (US)

Correspondence Address: ST. ONGE STEWARD JOHNSTON & REENS, LLC, 986 BEDFORD STREET, STAMFORD, CT, 06905-5619, US

(21) Appl. No.: 10/856,713

(22) Filed: May 28, 2004

Related U.S. Application Data
(63) Continuation-in-part of application No. 10/637,891, filed on Aug. 8, 2003.

(51) Int. Cl. A61K 35/78

(52) U.S. Cl. 424/726; 424/730; 424/737; 424/744; 424/757; 424/750; 424/764; 424/765; 424/779; 424/741

(57) ABSTRACT

A method of formulating a personal care and/or fragrance product comprises selecting a specific ABO blood type and using a group of product ingredients correlated as beneficial to the selected ABO blood type to formulate the personal care product. A personal care product for use by a human of a specific ABO blood type to prevent an antibody response comprises a plurality of ingredients selected to omit one or more of the A antigen and similar compounds and the B antigen and similar compounds.

### RELATED APPLICATIONS

The present application is a continuation-in-part of U.S. application Ser. No. 10/637,891 filed Aug. 8, 2003.

### FIELD OF THE INVENTION

The present invention relates to the field of formulation of personal care, cosmetic and/or fragrance products. More specifically, the present invention relates to personal care, cosmetic and/or fragrance products formulated to minimize allergic or other undesirable immune reactions, and methods of formulating thereof, based on the user's blood type to minimize the presence of isoantigen-like substances which trigger an antibody reaction in the user's specific blood type.

### BACKGROUND OF THE INVENTION

Cosmetics and skin care products typically include a number of different components selected to provide a desired consistency, coloring, fragrance, and shelf life. However, the formulation of these products requires a care to avoid potential allergic reaction in the user, typically a reaction of the skin to one or more components of the product. These reactions can range from minor inflammation to, in extreme cases, mast cell degranulation with associated symptoms of bumps, itching, redness etc. cause by the histamine release. The reactions vary among different individuals. The reactions vary with the specific ingredients of the cosmetic, and the amount or quantity used, and the sensitivity of the user to the formulation components and/or admixtures. While there are many personal care products that are so-called “hypoallergenic” compositions, they only address skin allergens and irritants. These compositions do not address the possibility of a reaction of immune system based upon an individual's ABO blood type, nor do they consider the potential of unnecessary immune system reaction and the production of isoagglutinins by the immune system.

I have observed certain relationships between human blood type, vitamin, mineral and herb intake, and illness and these observations were published in 1996 in my book entitled “Eat Right 4 Your Type.” This book, and my subsequent books disclose use of compositions and the benefit of selecting foods and supplements that are of benefit for particular blood type. Blood type specific vitamins and dietary supplements have been sold with my name since at least 1999.

There remains a need to formulate personal care products that are formulated based in part upon an individual's blood type to prevent a wide range of immune reactions that would otherwise decrease health of the skin and the individual overall.

### SUMMARY OF THE INVENTION

It is an object of the present invention to provide a method of formulating personal care, cosmetic and fragrance compositions, as well as personal care, cosmetic and fragrance compositions themselves, which reduce the incidence of allergic reaction or immune response causing isoagglutinin production.

It is an object of the present invention to provide a method of formulating personal care, cosmetic and fragrance compositions, as well as personal care, cosmetic and fragrance compositions themselves, which match the personal biochemical composition of the user.

It is an object of the invention to provide a method of formulating personal care, cosmetic and fragrance compositions, as well as personal care, cosmetic and fragrance compositions themselves, which are blood type specific, e.g. formulated particularly with the ABO blood type system.

These objects, and other objects as may become apparent hereafter, will be apparent from the following detailed description.

### DETAILED DESCRIPTION OF THE INVENTION

The human ABO blood group system is defined by the presence or absence of specific antigens in the blood. These unique carbohydrate or carbohydrate combinations found on the membrane of red blood cells (RBCs) define a person's blood type. RBCs having the carbohydrate n-acetyl galactosamine, the A antigen, are called blood type A. RBCs having the carbohydrate galactose, the B antigen, are called blood type B. RBCs of blood type AB have both the A and B antigens, e.g. both n-acetyl galactosamine and galactose. RBCs of blood type O have no antigens.

These RBC antigens are called isoantigens, which are proteins or other substances that are present in only some members of a species and therefore able to stimulate antibody production in other members of the same species who lack the antigen. Humans who are exposed to foreign isoantigens, and antigens very similar to isoantigens, produce antibodies that respond to the A and/or B antigens absent from their own RBCs. These are termed isoantibodies, and more specifically, isoagglutinins, the term for antibodies normally present in the sera of individuals that cause agglutination of the RBCs of another individual of the same species.

The immune system of a person of blood type A recognizes as foreign and will react to exposure to the B antigen, galatose, and produce anti-galactose antibody, called anti-B antibody or anti-B isoagglutinin. Likewise, the immune system if a person of blood type B will react to exposure to the A antigen, n-acetyl galactosamine, and will product anti-n-acetyl galactosamine antibody, called anti-A antibody or anti-A isoaglutinin. The immune system of a person of blood type AB will not react to exposure to either the A or B antigens, galactose or n-acetyl galactosamine, and produces no antibodies to them. In contrast, a person of blood type O recognizes both the A or B antigens, galactose or n-acetyl galactosamine, as foreign, and will produces both anti-A and anti-B antibodies/isoagglutinins.

The A and B antigens found in the molecules of human RBCs also exist in other biological entities, notably, bacterial cell walls, plants, and other foodstuffs. Bacteria are widespread in the environment, are present in intestinal flora, dust, food and other widely distributed agents, ensuring a constant exposure of individuals to A and B antigens and antigens that are extremely similar in structure to each of these isoantigens. This may explain why individuals who have not been otherwise exposed to the antigen, for instance to incompatible blood via transfusion, will have a detectable isoagglutinin level in the blood stream. Isoagglutinin production may be a reaction to environmental provocations of antigens. Small amounts of A and B antigens may enter the body in food, bacteria, or by other means, and these substances initiate the development of isoagglutinins, e.g. the anti-A antibodies and/or anti-B antibodies. See Guyton, A. C., Textbook of Medical Physiology 8^{th} ed., W. B. Saunders Co., 1990.

Isoagglutinin production is generally seen after the first few months of life and continues throughout an individual's life, remaining fairly constant until late in adult life. See Liu, Y J et. al., The development of ABO isohemagglutinins in Tawanese. Hum. Hered. July/August 1996, 46(4):181-4. In the elderly, isoagglutinin production has been found to diminish and it is believed that this is due to the gradual reduction in efficiency of the immune defenses as the cells age. Recent studies measuring isoagglutinin levels suggest that the baseline isoagglutinin levels in children have risen over time. See Godzisz, J., Synthesis of natural allohemagglutinins of the ABO blood system in healthy children aged 3 months to 3 years, Rev. Fr. Tranfus. Immunohematol., September 1979, 22(4): 399-412.

Studies have suggested that elevated issoagglutinin levels are linked with disease in humans, particularly those involving auto-immune dysfunction and/or excess inflammation, such as rheumatoid arthritis, endometriosis, chronic ear infection, Chrohn's disease of the intestines, asthma, eczema, and hives. See D'Adamo, P. Does ABO bias in innate immunity imply a different in T-cell reponse?, J. Naturopath. Med. 1991, 2:11-17.

It is possible that continual stimulation of the immune system by isoantigens and similar substances encountered by the body unnecessarily consumes the resources of an individual's immune system and eventually reduces the immune systems ability to defend against real threats such as viruses, bacteria, and other harmful foreign antigens, thereby degrading overall health.

I suggest that it is beneficial to overall health to avoid triggering an immune reaction where not required to respond to disease or other pathogens, or toxins, thus reducing production of antibodies and inflammation. The development of personal care and cosmetic products which are non-reactive to an individual's isoantigens could exert a resource-sparing effect on an individual's antibody defenses, prolonging immune competency over the course of his/her lifespan, modulating inflammation, improving fertility and enhancing host resistance. Further, personal care and costmetic product formulations may take into account the biochemical differences among different persons in their secretions and biochemistry, such differences being linked to blood type.

As used in this application, the term “personal care products” is used to identify the broad class of products applied topically to the body, as to the skin and hair both including products applied for purely cosmetic purposes as well as for therapeutic purposes. Thus the term “personal care products” as used herein, and the scope of the invention, extends to products such as (1) skin care products such as creams, lotions, skin toners, sun blocks, defoliants, with and without active ingredients such as alpha-hydoxy acids (for example glycolic acid), hydroquinone, octyl methoxycinnamate, etc.; (2) cosmetics such as lipstick, foundation make-up, blush, eye shadow, mascara, etc.; (3) personal care products such as soaps, shaving creams, and deodorant; (4) shampoos and conditioners; (5) non-skin care products topically applied to the body, such as eye drops, toothpaste, mouthwash, etc.; and (5) fragrance products, such as perfume, colognes, body sprays, eau de toilette, etc.

A predictive test for determining whether a user will react adversely when a personal care or cosmetic product begins with assessment of serologic parameters related to blood type and phenotypic expression of adjacent alleles; preferably, the assessment of serologic parameters comprises determining blood type using the ABO system. Allergenic reaction to each component and/or admixtures of components in a personal care product is then determined with reference to the user's blood type. The determination of allergenic reaction may begin initially with review of published medical and scientific literature, such as prior allergy studies, as well as naturopathic studies regarding the influence on the body, dependent on blood type, of various lectins present in foods or other sources.

In addition to negative information (i.e. what product components create an allergic reaction) it is anticipated that it will also be possible to develop positive information. Thus, for particular blood types, information will be developed as to what product components provide beneficial, therapeutic, and/or enhanced effects, such as reduction of inflammation. The positive and negative effect information will be compiled principally by determination of the effect of the product on the human tissue, principally the skin, or other intended area of the body to which the product is to be applied. However, if other data with respect to such effects is developed it may be so included.

The negative and positive information so developed will then be used to guide additional clinical studies, to check and/or test reaction of users of different blood types to the components and/or admixtures thereof in personal care products. A data base may then be created that provides, for each human blood type, a categorization of various possible components as beneficial/therapeutic, neutral, or potentially allergenic (or otherwise potentially harmful).

The possible components selected for investigation and inclusion in the database will include not only conventional ingredients for such products, but also other components not typically considered in to the formulation of personal care products. These may include products derived from seaweed or food products.

A typical formulation for sun block/skin lotion could include Octyl Methoxycinnamate, Zinc Oxide, Allantoin, Glyceryl Stearate, C12-15 Alkyl Benzoate, Glycerin, Steareth-2, Steareth-100, Tricontanyl PVP, PEG-100 Stearate, Tocopheryl Acetate, Xanthan Gum, Cetyl Hydroxyethylcellulose, Dimethicone, Disodium EDYA, Methylparaben, and Propylparaben. In a composition in accordance with the invention, blood type-specific ingredients might be removed or added to such a formulation to achieve the desired non-inflammatory response.

In a specific example of a composition in accordance with the invention, in an exfoliating cream using alpha hydroxy acids to achieve a skin peel, the product formulations would accommodate blood type specific reactions. Thus, an exfoliating cream suitable for users of Type O blood might not incorporate lactic acid, given the Type O blood types tend to be intolerant of such milk-derived compounds. Instead, other types of alpha-hydroxy acids, such as glycolic acid (derived from sugar cane), malic acid (from apples), tartaric acid (from grapes) or citric acid (from citrus fruit) would be used.

The above description principally addresses the prevention of undesired reactions to product formulations by consideration of the user's blood type; however, the invention also extends to creating product formulations taking account of the biochemical differences among different persons in their secretions and biochemistry, which are linked to blood type. For example, it is known that bacteria, both benign and harmful, will flourish to a greater or lesser degree according to blood type. This information could be used in the formulation of mouth was and/or toothpaste products to develop products for persons of different blood type to provide a more effective dental care program, to prevent the bacteria that causes gum disease and/or to achieve a more effective control of breath odors caused by the presence of bacteria. The same kind information could be used in connection with skin care products such as topical products for prevention of acne. ABO blood group antigens are extensively distributed in mucous, perspiration and saliva. Evidence suggests that the glycoprotein components of each ABH antigen serve as an energy substrate for different strains of Gram negative organisms. See Hoskins L C et al., Degradation of blood group antigens in human colon ecosystems. J. Clinical Invest. 1976; 57: 74-82; Hoskins L C. Et al. Mucin degradation in human colon ecosystems. Isolation and properties of fecal strains that degrade ABH blood group antigens and oligosaccharides from mucin glycoproteins. J. Clinical Invest. 1985:944-53) Thus the use of essential oils (in fragrance and personal care products) with anti-microbial or probiotic functions can serve to alter and optimize the microbial balance of the skin along lines predictable by ABO blood group.

It is also within the scope of the invention to use blood type information in the determination of fragrance products. Again, the human blood types may express various human pheronomes. It would be possible to develop and test various fragrance products that are more alluring or attractive to persons of specific blood type, to create interest in the wearer of the fragrance by the person receiving the sensory information. In the method in accordance with the invention pertaining to fragrance products, the steps will involve creating a data set defining negative or positive pheronome effects of a selected group of product ingredients as correlated to serologic parameters associated with blood type; selecting a blood type for which the said fragrance product will be formulated; and using ingredients in a formulation of said fragrance product, using product ingredients selected from said data set which are correlated to said selected blood type as lacking negative pheronome effects for such blood type or as having positive pheronome effects for such blood type. The formulation may be selected based on the blood type of the person who will wear the fragrance product, or a persons other than the wearer, e.g. the person who will sense and react to the product.

Accordingly, one aspect of the invention comprises personal care products for use by a human of a specific ABO blood type, said personal care and cosmetic product being selected and formulated to omit one or more of the A antigen and the B antigen and which would stimulate an antibody response in said human of specific ABO blood type. Providing personal care and cosmetic products that exclude substances which are foreign to and will provoke a immune response based upon an individual human's blood type and its resulting reactivity, will be a benefit to that individual's immune system, preventing unnecessary response and stress on the system, and will result in an overall improvement of skin health and overall well-being. In addition to not causing immune or allergenic reaction, substances may be beneficial to individual of specific ABO blood type as they are compatible and synergistic with the biochemistry of the specific ABO blood type and serve to promote overall health of the individual.

In one embodiment of the present invention, personal care products formulated to support and improve the health, in particular of the skin, of blood type O individuals and prevent production of anti-A and anti-B isoagglutinins exclude those substances, vitamins, minerals, herbals and other botanicals, amino acids, carbohydrates, oils, glycoconjugates, fatty acids, that are similar to the A- and B-isoantigens and therefore will prompt an immune response.

Thus Vitamin A and vitamin E are excluded from personal care products formulated for blood type O individuals according to the present invention. Herbals including alfalfa, aloe, burdock, coltsfoot, corn silk, Echinacea, gentian, goldenseal, red clover, rhubarb, Saint-John's wort, senna, shepherd's purse, strawberry leaf and yellow dock and oils including corn oil, cottonseed oil, peanut oil, and safflower oil are excluded from personal care products formulated to be nonreactive to blood type O individuals according to the present invention.

Personal care products to support and improve the health of persons of blood type O according to the present invention may contain vitamins, minerals, herbals and other botanicals, amino acids, carbohydrates, oils, glycoconjugates, and fatty acids and that are known non-reactive and with the B-isoantigen and biochemically compatible therewith.

Beneficial vitamins which are non-reactive to blood type O include vitamin B, specifically vitamin B-12 and folic acid, and vitamin K. Beneficial minerals known to be non-reactive to blood type O include calcium, manganese and iodine.

Beneficial herbs and phytochemicals known to be non-reactive to blood type O are licorice (Glycyrrhiza glabra), bladder wrack (Fucus vesiculosus) chickweed, dandelion, fengugreek, ginger, hops, linden, mulberry, parsley, peppermint, rose hips, sarsaparilla, and slippery elm.

Beneficial oils known to be non-reactive to blood type O are linseed oil and olive oil.

In another embodiment of the present invention, personal care products formulated to support and improve the health, particularly of the skin, of persons of blood type A and prevent production of anti-B isoagglutinin are designed to exclude those substances, vitamins, minerals, herbals and other botanicals, amino acids, carbohydrates, glycoconjugates, fatty acids, that are similar to the B isoantigen and therefore will prompt an immune response. Vitamin A, beta carotene is excluded from personal care products formulated for blood type A individuals according to the present invention.

Beneficial vitamins which are non-reactive to blood type A include vitamin B, particularly vitamin B-12, vitamin C, and vitamin E.

Beneficial minerals known to be non-reactive to blood type A provided by personal care products according to the present include calcium, particularly calcium lactate and calcium citrate, iron, and zinc.

Benefical oils known to be non-reactive to blood type A provided by personal care products according to the present include linseed oil and olive oil.

Beneficial herbs and phytochemicals known to be non-reactive to blood type A and beneficial to the immune system are hawthorn, Echinacea, in particular purple coneflower (Echinacea purpurea), and huangki (Astragalus membranaceous). Other known beneficial herbs non-reactive to blood type A include chamomile, valerian root, quercetin, milk thistle, bromelain, pro-biotic material (“good” bacteria high in bifidus factor), alfalfa, aloe, burdock, fenugreek, ginger, ginseng, green tea, rose hips, Saint-John's wort, slippery elm, and stone root.

In another embodiment of the present invention, personal care product formulated to support and improve the health of persons of blood type B and prevent production of anti-A isoagglutinin are designed to exclude those substances, vitamins, minerals, herbals and other botanicals, amino acids, carbohydrates, glycoconjugates, fatty acids, that are similar to the A-isoantigen and therefore prompt an immune reaction.

Personal care products to support and improve the health of persons of blood type B may contain vitamins, minerals, herbals and other botanicals, amino acids, carbohydrates, glycoconjugates, and fatty acids and that are known non-reactive with the A-isoantigen.

Beneficial vitamins nonreactive to blood type B include vitamins A, B, E, and C. Beneficial minerals which are non-reactive to blood type B include magnesium.

Beneficial herbs and phytochemicals known to be non-reactive to blood type B include licorice (Glycyrrhiza glabra), adaptogenic herbs such as, ginseng, specifically Siberian ginseng (Eleutherococcus senticosus) and Ginkgo biloba, licorice, ginger, parsley, peppermint, raspberry leaf, rose hips, sage, and lecithin (found in soy). Additionally, dietary supplements beneficial and non-reactive to blood type B individuals may contain enzymes, such as bromelain.

Herbals known to be reactive to blood type B individuals which are excluded from personal care products formulated for blood type B individuals according to the present invention include aloe, coltsfoot, corn silk, fenugreek, gentian, goldenseal, hops, linden, mullein, red clover, rhubarb, senna, shepherd's purse, and skullcap.

Oils known to be reactive to blood type B individuals which are excluded from personal care products formulated for blood type B individuals

In another embodiment of the present invention, personal care products to support and improve the skin health of persons of blood type AB may contain vitamins, minerals, herbals and other botanicals, amino acids, carbohydrates, oils, glycoconjugates, and fatty acids and that are known to be compatible with the biochemistry of blood type AB individuals. Further, such ingredients which are known to be bichemically incompatible with blood type AB individuals are omitted.

Blood type AB Incompatible ingredients are oils including corn oil, cottonseed oil, safflower oil, sesame oil, sunflower oil and herbals including aloe, coltsfoot, corn silk, fenugreek, gentian, hops, linden, mullein, red clover, rhubarb, senna, shepherd's pie, skullcap.

Beneficial vitamins which are non-reactive to blood type AB include vitamin A, vitamin B-12, niacin, vitamin E, and vitamin C, preferably from rose hips.

Beneficial herbs and phytochemicals known to be biochemically compatible with blood type AB individuals include hawthorn (Crataegua oxyacantha), purple cornflower (Echinacea purpurea), huang-ki (Astragalus membranaceous), chamomile, valerian root, quercetin, milk thistle (Silybum marianum), and bromelain.

Beneficial minerals which are non-reactive to blood type AB provided by personal care products according to the present invention include iron, zinc, and selenium.

Beneficial oils which are non-reactive to blood type AB provided by personal care products according to the present invention include olive oil.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention

1. A method of formulating a personal care product for use by a human of specific ABO blood type comprising the steps of:
selecting the specific ABO blood type for which the said personal care product will be formulated;
using a plurality of ingredients in a formulation of said personal care product which are correlated to said blood type as not stimulating isoagglutinin production in said human of said blood type.

2. A method of formulating a personal care product in accordance with claim 1, wherein said blood type is type A and said ingredients omit vitamin A, corn oil, cottonseed oil, peanut oil, safflower oil, sesame oil, catnip, cayenne, corn silk, red clover, rhubarb, and yellow dock

3. A method of formulating a personal care product in accordance with claim 1, wherein said blood type is type B and said ingredients omit Vitamin E, aloe, coltsfoot, corn silk, fenugreek, gentian, goldenseal, hops, linden, mullein, red clover, rhubarb, senna, shepherd's purse, and skullcap.

4. A method of formulating a personal care product in accordance with claim 1, wherein said blood type is type O and said ingredients omit vitamin A, vitamin E, corn oil, cottonseed oil, peanut oil, safflower oil, alfalfa, aloe, burdock, coltsfoot, corn silk, Echinacea, gentian, goldenseal, red clover, rhubarb, Saint-John's wort, senna, shepherd's purse, strawberry leaf and yellow dock.

5. A method of formulating a personal care product in accordance with claim 1, wherein said blood type is type AB and said ingredients omit corn oil, cottonseed oil, safflower oil, sesame oil, sunflower oil, aloe, coltsfoot, corn silk, fenugreek, gentian, hops, linden, mullein, red clover, rhubarb, senna, shepherd's pie, skullcap.

6. A method of formulating a personal care product in accordance with claim 1, wherein said step of using ingredients further comprises the step of using ingredients which are correlated to said blood type as having positive biochemical effects for said human of said blood type.

7. A method of formulating a personal care product in accordance with claim 6, wherein said blood type is type A and said ingredients are vitamin B, vitamin C, vitamin E, calcium, iron, zinc, selenium, chromium, linseed oil, olive oil, afalfa, aloe, burdock, chamomile, Echinacea, huangki, quercetin, bromelain, fenugreek, ginger, ginseng, green tea, hawthorn, milk thistle, rose hips, Saint-John's wort, slippery elm, stone root, valerian, or any combination thereof.

8. A method of formulating a personal care product in accordance with claim 6, wherein said blood type is type B and said ingredients are magnesium, lecithin, licorice, olive oil, ginger, ginseng, Ginkgo biloba, licorice, licorice root, parsley, peppermint, raspberry leaf, rose hips, sage, and green tea, or any combination thereof.

9. A method of formulating a personal care product in accordance with claim 6, wherein said blood type is type O and said ingredients are vitamin K, vitamin B, calcium, iodine, linseed oil, olive oil, licorice, manganese, bladder wrack, or any combination thereof.

10. A method of formulating a personal care product in accordance with claim 6, wherein said blood type is type AB and said ingredients are vitamin C, zinc, hawthorn, Echinacea, huangki quercetin, milk thistle, bromelain, alfalfa, burdock, chamomile, ginger, ginseng, green tea, licorice root, rose hips, strawberry leaf, olive oil, or any combination thereof.

11. A personal care product for use by a human of a specific ABO blood type to prevent an antibody response, comprising a plurality of ingredients selected to omit one or more of the A antigen and similar compounds and the B antigen and similar compounds.

12. The personal care product in of claim 11, wherein said blood type is type A and said ingredients omit vitamin A, corn oil, cottonseed oil, peanut oil, safflower oil, sesame oil, catnip, cayenne, corn silk, red clover, rhubarb, and yellow dock.

13. The personal care product of claim 11, wherein said blood type is type B and said ingredients omit Vitamin E, aloe, coltsfoot, corn silk, fenugreek, gentian, goldenseal, hops, linden, mullein, red clover, rhubarb, senna, shepherd's purse, and skullcap.

14. The personal care product of claim 11, wherein said blood type is type O and said ingredients omit vitamin A, vitamin E, corn oil, cottonseed oil, peanut oil, safflower oil, alfalfa, aloe, burdock, coltsfoot, corn silk, Echinacea, gentian, goldenseal, red clover, rhubarb, Saint-John's wort, senna, shepherd's purse, strawberry leaf and yellow dock.

15. The personal care product of claim 11, wherein said ingredients further comprise substances which are correlated to said blood type as having positive biochemical effects for said human of said blood type.

16. The personal care of claim 15, wherein said blood type is type A and said ingredients are vitamin B, vitamin C, vitamin E, calcium, iron, zinc, selenium, chromium, linseed oil, olive oil, afalfa, aloe, burdock, chamomile, Echinacea, huangki, quercetin, bromelain, fenugreek, ginger, ginseng, green tea, hawthorn, milk thistle, rose hips, Saint-John's wort, slippery elm, stone root, valerian, or any combination thereof.

17. The personal care product of claim 15, wherein said blood type is type B and said ingredients are magnesium, lecithin, licorice, olive oil, ginger, ginseng, Ginkgo biloba, licorice, licorice root, parsley, peppermint, raspberry leaf, rose hips, sage, and green tea, or any combination thereof.

18. The personal care product of claim 15, wherein said blood type is type O and said ingredients are vitamin K, vitamin B, calcium, iodine, linseed oil, olive oil, licorice, manganese, bladder wrack, or any combination thereof.

19. The personal care product of claim 15, wherein said blood type is type AB and said ingredients are vitamin C, zinc, hawthorn, Echinacea, huangki quercetin, milk thistle, bromelain, alfalfa, burdock, chamomile, ginger, ginseng, green tea, licorice root, rose hips, strawberry leaf, olive oil, or any combination thereof.

20. A method of formulating a fragrance product, comprising the steps of:
creating a data set defining negative or positive pheronome effects of a selected group of product ingredients as correlated to serologic parameters associated with blood type;
selecting a blood type for which the said fragrance product will be formulated;
using ingredients in a formulation of said fragrance product, using only product ingredients selected from said data set which are correlated to said selected blood type as lacking negative pheronome effects for such blood type.

21. A method of formulating a fragrance product in accordance with claim 20, wherein said step of using ingredients further comprises the step of using product ingredients selected from said data set which are correlated to said selected blood type as having positive pheronome effects for such blood type.

22. A method of formulating a fragrance product, comprising the steps of:
creating a data set defining negative or positive pheronome effects of a selected group of product ingredients as correlated to serologic parameters associated with blood type;
selecting a blood type for which the said fragrance product will be formulated;
using ingredients in a formulation of said fragrance product, using product ingredients selected from said data set which are correlated to said selected blood type as having positive pheronome effects for such blood type.

23. A method of formulating a fragrance product in accordance with claim 22, wherein said step of using ingredients further comprises the step of using product ingredients selected from said data set which are correlated to said selected blood type as lacking negative pheronome effects for such blood type.

24. A method of formulating a fragrance product in accordance with claims 20, 8, 9, or 10, wherein said blood type is the blood type of a person wearing said ingredients.

25. A method of formulating a fragrance product in accordance with claims 20, 8, 9, or 10, wherein said blood type is the blood type of a person other than the person wearing said ingredients.
